(19) United States
(12) Patent Application Publication
(10) Pub. No.: US 2010/0099940 A1
(43) Pub. Date: Apr. 22, 2010

(54) Thulium-containing low dose rate brachytherapy seed

(75) Inventors: Gad Shani, Omer (IL); Amal Ayoub, Be'er Sheva (IL)

Correspondence Address: NIXON & VANDERHYE, PC, 901 NORTH GLEBE ROAD, 11TH FLOOR, ARLINGTON, VA, 22203, US

(73) Assignee: BEN-GURION UNIVERSITY OF THE NEGEV RESEARCH AND DEVELOPMENT AUTHORITY, Be'er-Sheva (IL)

(21) Appl. No.: 12/585,307

(22) Filed: Sep. 10, 2009

Related U.S. Application Data
(63) Continuation-in-part of application No. PCT/IL2008/000338, filed on Mar. 12, 2008.
(60) Provisional application No. 60/906,204, filed on Mar. 12, 2007.

(51) Int. Cl. A61M 36/12

(52) U.S. Cl. 600/8

(57) ABSTRACT

A low dose rate brachytherapy seed having activity in the range between 0.5 and 10 millicurie, preferably less than 3 millicurie, comprising thulium-170 placed within a casing and at least one layer of a radiation emission modifying metal, said layer being provided either internally within the casing or on the outer surface thereof. The radiation emission modifying metal is preferably gold.

Brachytherapy is a cancer treatment method in which ionizing radiation source is inserted into, or adjacent, a cancerous tumor, in order to kill cancer cells or shrink tumors. There are two common Brachytherapy treatment types: i) High Dose Rate (HDR); and ii) Low Dose Rate (LDR), which are distinct from one another by the intensity of the radiation source, its form and its method of use.

In LDR brachytherapy, low activity sources (also referred to as seeds or capsules), having diameter of about 0.8 mm and length of about 4.5 mm, are inserted into the tumor for a long period of time, practically till the activity of the radiation source decays completely. The activity of the LDR seeds is of the order of 0.5 millicurie (mCi). In order to obtain the radiation dose needed for killing all malignant cells in the tumor, a large number of seeds (100-120) are simultaneously implanted into the tumor.

Low dose rate brachytherapy is more or less limited to prostate cancer treatment while HDR brachytherapy is applied to other kinds of cancer too. Current LDR brachytherapy is also limited to tumors smaller than 50 cc, because of the large number of seeds required.

At present LDR brachytherapy is carried out using seeds containing the isotopes 1125 or Pd 103. I 125 emits photons of energy of about 28 keV and has a half life of 60 days. Pd 103 emits photons of energy of about 20 keV and has a half life of 17 days. It should be noted that the production of I 125 and Pd 103 is complicated, requiring the use of accelerator irradiation, isotope separation and hot laboratories for carrying out the radiochemistry of the seed production.

It has been proposed that thulium 170 may be used in brachytherapy. For example, WO 03/063757 describes a low dose brachytherapy seed, comprising the radioactive isotope thulium 170 placed within a titanium canister.

EP 1529554 describes a radioactive radiation source for ophthalmic brachytherapy. The radiation emitting element, which is preferably selected from the group consisting of Y-90, Tl-204, P-32 and Tm-170, is placed within a suitable tubular casing together with a shielding section made of metallic, ceramic or a plastic material. Among the metals proposed for preparing the shielding section, Pt, Pd, Au, Ag, Ir, Pb and W, together with their alloys, are specifically listed. The publication exemplifies the preparation of a radiation source comprising an oxide of yttrium-90 dispersed within an aluminum matrix, placed in a first metallic cylinder made of stainless steel. The first, stainless steel cylinder is inserted into a second cylinder made from Pt/Ir alloy. The structure thus obtained is finally sealed within a titanium cover.

International patent application no. PCT/IL2007/001196 discloses a capsule for high dose rate brachytherapy, wherein the capsule comprises within its interior space thulium-170, and further comprises at least one layer of a radiation emission modifying metal, and specifically gold, wherein said layer is provided either internally within the capsule or on the outer surface thereof. The activity of the radiation source within the seed is not less than 3 curie.

The inventor has found that the isotope thulium 170 (Tm-170), may be effectively used as a radiation source for low dose rate brachytherapy, in combination with a radiation emission modifying metal, which is preferably selected from the group consisting of gold (Au), platinum (Pt) and cerium (Ce). It has been found that the radiation emitted from thulium 170, which is composed of beta radiation (electrons) and gamma radiation (photons), may be favorably modified by coating the thulium 170 with one or more of the heavy metals listed above, and more specifically with gold.

According to the present invention, the radiation source includes a radioactive section, which comprises thulium 170, and at least one layer of a metal coating (specifically a gold coating) provided on the radioactive section. Although the primary purpose served by the heavy metal coating is to reduce the intensity of the beta rays emission from the thulium, the metal coating concurrently increases the intensity of the gamma radiation emitted by the source through two distinct effects: a braking radiation (bremssrahlung) effect in the metal coating, in which a part of the beta radiation turns into photons, and in addition, photons are also emitted from the metal coating in the form of characteristic x-rays, in response to absorption of some of the thulium 84 keV gamma rays. These effects are described in detail below.

Accordingly, by appropriately adjusting the thickness of the metal coating onto the thulium, it is possible control the intensities of the beta and gamma radiations emitted therefrom, generating a Tm-170 based low dose rate brachytherapy radiation source, characterized in that:

(i) the level of beta radiation emitted therefrom is reduced in comparison with that generated by Tm-170 in the absence of a radiation emission modifying metal;
(ii) the photon emission spectrum of said source displays a lower ratio between the intensities of the characteristic peaks at 84 keV and 52 keV energy levels, relative to the ratio observed in the corresponding spectrum of Tm-170 in the absence of a radiation emission modifying metal;
(iii) the photon emission spectrum of said source exhibits one or more peaks in the region between 28 kev and 84 keV energy levels, or preferably between 65 keV and 84 keV energy levels, indicative of photons emitted by the layer(s) of the radiation emission modifying metal;
(iv) the photon emission spectrum of said source comprises in addition to the discrete peaks also a continuous region indicative of the emission of braking radiation associated with the layer(s) of the radiation emission modifying metal.

The present invention thus primarily relates to a low dose rate brachytherapy seed, which comprises thulium-170 placed within a casing, wherein said seed further comprises at least one layer of a radiation emission modifying metal, said layer being provided either internally within the casing or on the outer surface thereof. Seeds having activity in the range between 0.5 and 10 millicurie may be utilized according to the invention. The activity of the seed is preferably not more than 3 millicurie, and more preferably between 0.5 and 2.5 millicurie.

The low dose rate brachytherapy seed of the invention comprises a casing or shell for containing a radioactive source. The casing may be made from any human tissue—compatible metal having a low atomic weight, such as titanium or stainless steel. Titanium or alloys thereof are especially preferred. The casing is preferably in the form of a cylindrical canister having the following characteristic dimensions: a length in the range between 4 and 7 mm, preferably about 4.5 mm; an outer diameter of about 0.5-1.0 mm, preferably about 0.8 mm; and an inner diameter of about 0.3-0.9 mm, preferably about 0.7 mm, such that the thickness of the wall of the cylindrical canister is about 0.05 mm. A relatively thin wall is important for the utilization of the beta rays generated by the thulium isotope. A thicker wall will absorb a certain percent of the beta rays. The outer diameter of the seed and its length as set forth above are as acceptable for LDR brachytherapy. The metal of which the canister is made is of course different from the radiation emission modifying metal to be applied in accordance with the invention. Hereinafter, the terms titanium casing, titanium canister or titanium capsule are used interchangeably.

Within the interior of the casing, the thulium 170 segment is placed. The seed preferably comprises a gold, platinum, iodine, barium, lanthanum or cerium coating which at least partially, and preferably entirely, surrounds the thulium 170 segment, said coating being either interposed between the radioactive isotope and the inner walls of the casing, or alternatively, applied onto the outer faces of the casing.

The seed of the present invention may be produced, for example, by coating the natural thulium 169 with one or more layers of a radiation emission modifying metal (e.g., gold), placing the coated thulium 169 in a casing as described above, e.g., a titanium casing, sealing the casing and subjecting the same to a neutron activation in a nuclear reactor in order to convert the thulium 169 into the radioactive isotope thulium 170 with the desired activity. Alternatively, the radiation emission modifying metal coating is applied onto the outer faces the casing, either prior to or following the activation of the seed in a nuclear reactor. The activation parameters for transforming the natural thulium 169 into thulium 170 are set forth below.

The metal coating, and especially the gold coating, is applied directly onto the thulium, or onto the outer surface of the casing, by means of various techniques. The thickness of the radiation emission modifying metal coating is preferably between a few atomic layers and up to 0.15 mm, and more preferably in the range between 0.02 and 0.1 mm. The thickness of the coating may be specifically tailored in order to adjust the radiation profile of the seed (namely, to determine the proportions of the beta and gamma radiation generated by the seed). One possible technique is electroplating, wherein the thulium is immersed in a solution containing gold ions and an electric current is passed through the solution to deposit a gold coating onto a thulium piece. Electroplating conditions (suitable gold solutions, medium stirring rates and current densities required for obtaining desired gold coatings) are known in the art. A second method is by gold evaporation. Heat is applied to a gold wire or rod (generally by electric current). Due to the high temperature gold atoms are evaporated and deposited on the thulium.

A further technique for making the gold coating is known as “spattering”, and has been found to be especially useful for forming relatively thin gold coatings onto the thulium (from 10 nm and up to 1 μm). A gold electrode and the thulium piece to be coated are positioned in a closed chamber and are separated by a distance in the range of 3 and 8 cm. The chamber is maintained under argon atmosphere at a low, constant pressure. A voltage of the order of 1-2 kV is applied between a gold electrode (directly connected to a power source) and the thulium piece to be coated. The electric field generated in the chamber ionizes the gas and causes a current to flow into the gold electrode. This results in gold being spattered from the electrode and being deposit onto the thulium piece. The thickness of the gold coating thus formed onto the thulium is given by the following equation:

Gold Thickness[Å]=k•I•t

Where I is the chamber current in mA, t is the spattering time in minutes and k is a constant characteristic of the device employed for the spattering.

The simplest technique for forming a gold, lanthanum or cerium coating is by wrapping the thulium piece with one or more gold, lanthanum or cerium foils of a desired thickness. Thin foils of said metals, of various thicknesses, are commercially available (Goodfellow, England). Iodine layer can be obtained by painting the thulium with iodine or immersing it in liquid iodine.

The amount of thulium placed in the seed according to the invention is preferably not less than 2 mg, and more preferably in the range between 2 and 8 mg. It is noted that thulium 169 to be converted according to the present invention to the radioactive thulium 170 is a readily processed metal, such that the final radioactive segment within the seed may attain various desired geometries, as illustrated in FIGS. 1, 2 and 3.

With reference to FIG. 1, a low dose brachytherapy seed 10 comprises a titanium tube 11 having the dimensions set forth above. The natural thulium 169 is commercially available in the form of a wire of a small diameter, typically about 0.45-0.65 mm. A piece of the wire, having length of about 1.0-4.0 mm, is coated with gold by means of the techniques described above (most simply by wrapping the thulium piece). Most conveniently, one of the open bases of tube 11 is closed with a plug 12a, and then the coated thulium rod 12 is introduced into the titanium tube through its open base, such that a thulium 169 rod 12t with a gold or other coating 12g provided thereon is affixed within the interior space 11i of the tube. It should be noted that the gold or other coating is provided on the lateral surface of the thulium 169 rod 12t and on its two bases. After placing the coated thulium section 12 in tube 11, the open base of tube 11 is sealed by a plug 12b. The plugs 12a, 12b are preferably made from one of the materials indicated above for tube 11, preferably from titanium. The diameter of the plugs 12a, 12b is adapted to snugly fit into tube 11, and their length is typically about 0.5 mm. After sealing tube 11 with plug 12b and following laser welding, the radiation source may be activated in a nuclear reactor by means of a neutron flux as described herein below. For considerations of safety, the seed 10 is delivered within a quartz ampoule to the nuclear reactor.

Alternative geometry of the radiation source 22 used in a brachytherapy seed 10 is shown in FIG. 2, where the thulium section 22t has the shape of rectangular parallelpiped having the following preferred dimensions: length between 1.0 and 4.0 mm, more preferably about 4 mm; width between 0.2 and 0.6 mm, more preferably about 0.6 mm; height between 0.2 and 0.4 mm. The rectangular parallelpiped may be obtained either by using one thulium 169 sheet with the desired shape and size, or by stacking together a plurality of thin thulium 169 sheets 22y, i.e., each sheet being 0.1 mm thick. Thulium sheets are commercially available (Goodfellow, England), and may be readily cut to the desired dimensions using a suitable punch. The thulium section is coated with gold 22g, introduced into the titanium tube 11 and sealed as described above with reference to FIG. 1. The sealed capsule may be delivered to a nuclear reactor for activation.

FIG. 3 illustrates an embodiment of the invention which is similar to the embodiment shown in FIG. 1, with the thulium section having a cylindrical symmetry. However, in this case the thulium section 12t is provided by a hollow rod, namely, a tube-like member defined by a lateral surface of a cylinder, rather than the solid rod exemplified to FIG. 1. The thickness of the walls of the thulium tube according to this embodiment is in the range of 0.1-0.2 mm (the thulium tube may be prepared by folding a thulium sheet into a tubular body). The thulium tube can be coated with gold on its outer surface, 12g, or the gold plating is applied externally onto the outer surface of the titanium capsule, rather than being internally placed. Following sealing as described above, the capsule may be delivered to a nuclear reactor for activation.

As mentioned above, the sealed capsule, with the natural thulium (Tm169) and the radiation emission modifying metal coating, is subjected to neutron activation at a nuclear reactor. The number of Tm170 atoms produced by the neutron activation is relative to the number of Tm169 atoms being activated. The number of Tm170 atoms obtained in the neutron activation equals the number of Tm169 atoms irradiated, times the neutron fluence times the activation cross-section. (Neutron fluence=neutron flux times activation time). The activity (number of disintegrations per second) of the Tm170 source, is given by the number of Tm170 atoms times the decay constant. Table 1 shows the activation time, in hours, at flux of 10¹³ n/s·cm², for three different geometries of the thulium section and four different activities. The number of Tm170 atoms which will provide the respect activity is also given in table 1. It is preferred to activate the thulium at a neutron flux of the order of 10¹³ n/cm²s so that the activation time is not too long (up to 24 hours). The activation time must be such that the desired activity is obtained, as explained above. This time in hours is given in table 1. Having obtained the required activity, the seeds must stay for about two weeks under shield for the heavy metal activity to decay.

TABLE 1
Time for activation to the given activity, in
hours, at flux of 10¹³ n/s · cm²

| 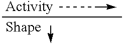 | 0.5 mCi | 1.0 mCi | 1.5 mCi | 2.0 mCi |
|---|---|---|---|---|
| Cylinder | 2.2 | 4.4 | 6.6 | 8.8 |
| 0.4 mm thick | 2.59 | 5.17 | 7.76 | 10.35 |
| rectangular | | | | |
| parallelpiped | | | | |
| 0.2 mm thick | 5.18 | 10.35 | 15.5 | 20.7 |
| rectangular | | | | |
| parallelpiped | | | | |
| Number of | 2.95 × | 5.9 × 10¹⁴ | 8.86 × 10¹⁴ | 1.18 × 10¹⁵ |
| Tm170 | 10¹⁴ | | | |
| Atoms for | | | | |
| Activity | | | | |

The Tm170-containing seed obtained following the activation described above emits both beta and gamma rays and therefore it can be used for treatment of small tumors as well as large tumors. The beta rays may be used for treating small tumors, while the gamma photons are more suitable for treating large tumors. According to a preferred embodiment of the invention, the radiation emission modifying metal completely surrounds the thulium and has a substantially uniform thickness. By the term “substantially uniform thickness” is meant that the tolerance in the thickness of the coating is not more than ±10%. Thus, the brachytherapy seed of the invention is essentially isotropic, namely, the radiation emitted therefrom has essentially the same intensity regardless of the direction of measurement. The intensity of the isotropic beta radiation generated by the seed is more than 10%, and preferably in the range between 10% and 30%, and more preferably in the range between 20 and 30%, relative to the intensity of the beta radiation emitted by a corresponding non-coated thulium 170. Changes in the emission of beta radiation can be determined by applying a radiation detector, either a thin window ion chamber or a scintillator.

The range of the 970 keV beta rays, which comprises 76% of the beta rays emitted from the Tm170, is about 3 mm. However, the presence of a metal (gold) coating of thickness of between few atomic layers (e.g., few Angstroms) to about 0.15 mm favorably modifies the radiation emission profile of the source, in cases wherein the beta rays are not wanted or their intensity should be lowered. Specifically, in case of a thick heavy metal layer (e.g., ˜0.15 mm), all beta radiation of energy of up to 1 MeV is blocked. The intensity of the photon radiation in this case is increased due to the bremssrahlung effect in the heavy metal coating layers in which a part of the beta radiation turns into photons. The radiation emission profiles of the modified source according to the present invention will now be discussed in more detail with reference to FIGS. 4 to 6. In these figures the abscissa is the energy of the emitted photon and the ordinate is the intensity, recorded by multychannel analyzer. Photon spectrum measurements may be carried out using a conventional gamma spectrometry system, which includes: a gamma solid state detector, either HPGe or Si(Li), a high voltage supply, preamplifier, amplifier and a multychannel analyzer. Changes in the emission of beta radiation can be determined by applying a radiation detector, either a thin window ion chamber or a scintillator.

The radiation emitted from Tm 170 is composed of beta radiation (electrons) and gamma radiation (photons). 76% of the beta radiation is of energy Eₘₐₓ=968 keV and 24% of it is of energy Eₘₐₓ=884 keV. FIG. 4 shows the characteristic photon spectrum of Tm 170 (non-coated). As seen in FIG. 4, the photon spectrum has a main peak at 84 keV energy level and other smaller peaks, in the energy range 50-60 keV. More specifically, there are three photon energies emitted from Tm170, the first is composed of gamma rays having a 84 keV energy level emitted from excited Y170 atom formed upon the beta decay of Tm170, and the other two are x-rays at about 50-60 keV energy level, emitted following the rearrangement of the Y170 electrons, after the interaction of some of the photons emitted from the nucleus, with the atomic electrons. The ratio of gamma to x-ray emission intensity is typically about 3:1.

When the beta rays emitted from the Tm 170 enter the heavy metal layer (e.g., gold) covering the thulium, most of them are stopped by collision with the electrons of the heavy metal atoms. This is the main beta removal effect caused by the heavy metal. A small fraction, 2-5% as shown in table 2 below, undergo the effect known as braking radiation (Bremsstrahlung in German) due to deceleration of these electrons in the electric field of the heavy metal electrons or the heavy metal nuclei. The energy of those beta rays is turned into photons of continuous spectrum, mostly in the lower energy range. The fraction of the beta ray turning into photons by braking radiation is proportional to the beta energy times the atomic number of the stopping material. The fraction of the beta intensity turning into photons and the photons energy obtained for each beta ray are shown in table 2 for three possible heavy metals: platinum, gold or lead. The columns marked by the % symbol in table 2 indicate the percent of the beta rays turned into photons in the braking radiation process, for each beta energy. The columns entitled by “Energy” give the photon energy obtained from the beta rays, for each beta energy. The columns entitled by “76%” and “24%” give the actual photon energy obtained, from each beta energy. The last column is the sum of the two, i.e. total photon energy obtained from the beta rays by braking radiation.

TABLE 2
Photon fractions added to the spectrum due to Braking Radiation

| | | | Eₘₐₓ = 968 keV | | | Eₘₐₓ = 884 keV | | | Total max |
|---|---|---|---|---|---|---|---|---|---|
| | Atomic | d | | E | 76% | | E | 24% | energy Per |
| Metal | Number | gm/cm³ | % | [keV] | [keV] | % | [keV] | [keV] | Disintegration |
| Pt | 78 | 21.45 | 2.64 | 25.6 | 19.44 | 2.41 | 21.33 | 5.12 | 24.56 keV |
| Au | 79 | 19.3 | 2.68 | 25.9 | 19.69 | 2.44 | 21.6 | 5.19 | 24.88 keV |
| Pb | 82 | 11.35 | 2.78 | 26.9 | 20.44 | 2.45 | 22.43 | 5.38 | 25.82 keV |

It should be noted that the activation of the seed may be carried out either prior or subsequent to the incorporation of the radiation emission modifying metal within the seed. In the event that the coating is applied in the seed before the activation, namely, a titanium casing which comprises Tm 169 and a gold or platinum layer is activated by neutrons to get the radioactive isotope Tm 170, then the coating material is activated too and a radioactive isotope thereof is formed. The activation of the titanium is negligible. These radioactive isotopes are short lived: T1/2Pt195=4 days, T1/2Au198=65 hours, T1/2Pb209=3.3 hours. The respective activation cross sections are: σPt=10 b, σAu=98.8 b and σPb=0.17 b. The activity of these isotopes is undesired in the radiation source, therefore, after being activated the radiation source should stay in the reactor area for about two weeks cooling period for these isotopes to decay. The two weeks decay time has a minor effect on the Tm 170 activity; it decays to 93% of its activity at the end of the activation.

The heavy metal coating (such as Au, Pt, Bi, Tl or amalgam of Hg, with gold being especially preferred) surrounds the thulium core in the seed of the present invention and forms part of the radiation source. Although the heavy metal coating is not radioactive, it emits radiation as a result of absorbing the radiation from the Tm 170 core. The heavy metal coating applied over the radiation source reduces, or stops completely (depends on its thickness), the beta rays emitted from the Tm 170 core, from leaving the radiation source into the tumor. A part of the beta rays absorbed by the heavy metal coating the Tm turns into photons as braking radiation. An additional photon source is created due to x-ray emitted from the heavy metal coating, this x rays emission is typical to those metals. These x rays energies are in the range of 66-84 keV respective to the metal emitting it.

Thus, the metal coating the Tm 170 core of the radiation source changes the radiation emitted therefrom in a number of ways. The effect it has on the beta rays is the most drastic because this thin coating either eliminates the beta rays emission completely or reduces it to a desirable degree. The thicker the coating layer the weaker is the beta intensity.

The second effect of the heavy metal coating layer on the radiation emitted from the radiation source is the addition of a continuous x ray spectrum, to the photon emitted from the radiation source. Stopping the beta radiation by the heavy metal causes the emission of braking radiation. This is a continuous photon spectrum of energy from very low (1-3 keV) to the highest energy of the beta rays-Emax emitted from the Tm 170, as can be seen in FIG. 5.

The third effect of the heavy metal coating on the photon spectrum emitted from the radiation source is the additional peaks in the spectrum, related to x rays emitted from said metal coating. The source of these x rays is the photoelectric effect caused by the 84 keV photons emitted from the Tm 170. The main x ray peaks are at energy levels: 67, 69, 78 and 80.1 for gold; 65, 67, 75.7 and 77.8 for platinum. The main gold peaks are seen in FIG. 5.

The photoelectric effect caused by the 84 keV photons lowers this peak in the spectrum as can be seen in FIG. 5. (The ratio between the 84 keV peak and the 52.4 keV peak has been reduced compared to that seen in FIG. 4; according to the modified source of the present invention, the ratio between said peaks is less than 2.5:1). This is fourth spectrum change due to the heavy metal coating the TM 170.

To sum up, the changes in the photon spectrum caused by the heavy metal layer are: additional low energy photons, reduction in the 84 keV peak and addition of x rays peaks corresponding to the metal coating the thulium radiation source. The change in the beta spectrum: lowering its intensity or complete removal.

The addition of the low energy photons to the spectrum increases the efficiency of the radiation source at small distance. Although lowering the 84 keV photons peak reduces the number of photons that can reach large distance from the source, this is sufficiently compensated by the addition of the higher energy x rays emitted by the heavy metal and by the higher photons in the braking radiation.

FIG. 6 (comparative) shows the photon spectrum of a Tm170 radiation source in which the radioactive section was not coated by gold but encased in a titanium seed having wall thickness of about 0.05 mm. The continuous spectrum beyond the 84 keV peak is due to braking radiation in the titanium.

In FIG. 7, the radiation dose associated with the gamma emission generated by 2 mCi Tm 170 seed, wherein the thulium is, coated with 0.1 mm gold foil, is plotted as a function of distance (mm) from the source. The radiation dose was measured with LiF(Mg,Ti) TLD in Lucite (PMMA) phantom. The units of the ordinate are millirem per hour.

In another aspect, the radiation emission modifying element used belongs to a class of intermediate weight elements, having atomic number in the range between 53 and 58, wherein said element is preferably selected from the group consisting of iodine, lanthanum, barium and cerium. The elements indicated above are capable of converting the high energy photons generated by thulium 170 into lower energy photons due to the photoelectric effect. More specifically, by using said elements, and especially cerium, the 84 keV and the 48-52 keV complex photons generated by thulium 170 are converted into x-rays in the range 28-40 keV. Thus, the purpose of using the intermediate weight metal converter is to better utilize the photon energy emitted from the thulium 170 source. This is for the reason that the range of the 84 keV photons in tissue is about 6 cm and that of 50 keV is about 4.5 cm, while the range of 35 keV photons is smaller (about 3 cm). This small range, however, may suffice for certain tumors, such as prostate tumors.

The intermediate weight metal coating responds to the 84 and the 50-52 keV photons emitted from the Tm 170, generating x-ray characteristic of the coating metal. The characteristic x-ray generated by each one said elements is given in the following table:

TABLE 3
Characteristic x-ray of the intermediate weight metals

| The element | The characteristic peaks (keV) | | | |
|---|---|---|---|---|
| I | 28.3 | 28.6 | 32.3 | 33.0 |
| Ba | 31.8 | 32.2 | 36.4 | 37.3 |
| La | 33.0 | 33.4 | 37.8 | 38.7 |
| Ce | 34.3 | 34.7 | 39.2 | 40.2 |

Regarding the activation of the intermediate weight metals listed above, the thermal neutron activation cross section of those metal is the following in units of barns: I-1.5b, Ba-1.2b, La-0.064b and Ce-0.73b.

The half-life of the main product: 1128-25 min, Ba 139-83 min, La 140-40 h and Ce 141-32.5 d.

The isotopes of I, Ba and La are very short lived and will not affect the radiation emitted from the Tm-170 seeds. Ce 141 emits 145 keV gamma photons, but due to its low activation cross section and the small amount of the metal in the seed, its effect is negligible relative to that of Tm 170.

The seed according to the present invention is deliverable to the tumor site by means of commonly applied applicators (e.g., catheters, needles) in accordance with low dose rate brachytherapy protocols. For example, in the case of prostatic cancer, one possible technique involves loading the seeds into the cannula of a needle-like insertion device. Improved techniques for implanting brachytherapy seeds, which may be practiced according to the present invention are disclosed, for example, in U.S. Pat. No. 6,036,632, U.S. Pat. No. 6,267,718 and U.S. Pat. No. 6,311,084. Further references describing brachytherapy protocols are the following books:

Radiation Aspects of Brachytherapy For Prostate Cancer. ELSEVIER, July 2006.

Brachytherapy Oncology Update 1984, B. S. Hilaris and D. Nori, ed. Memorial Sloan-Kettering Cancer Center, 1275 York Avenue, New York, N.Y. 1984.

Accordingly, the present invention further provides a low dose rate brachytherapy method, which comprises implanting into or adjacent a tumor site of a patient a plurality of low dose rate thulium 170 brachytherapy seeds, wherein the thulium brachytherapy seed is provided with a coating of a radiation emission modifying metal, the activity of the seed being in the range between 0.5 and 10 millicurie, and preferably less than 3 millicurie, whereby beta radiation and gamma radiation, which are preferably isotropic, are delivered to said tumor. The tumors that can be treated with the seeds of the present invention include prostate, breast, various gynecological and head and neck tumors. The number of seeds to be implanted is determined according to the type and size of the tumor, and may vary in the range between 20 and 100. The seeds are removed from the body of the patient after the activity of the radiation source decays completely (about six months).

### BRIEF DESCRIPTION OF THE DRAWINGS

The present invention is illustrated by way of example in the accompanying drawings, in which similar references consistently indicate similar elements and in which:

FIG. 1 illustrates one embodiment of the LDR brachytherapy seed of the invention;

FIG. 2 illustrates another embodiment of the LDR brachytherapy seed of the invention;

FIG. 3 illustrates another embodiment of the LDR brachytherapy seed of the invention.

FIG. 4 (comparative) shows the characteristic photon spectrum emitted by Tm-170;

FIG. 5 shows a photon energy spectrum measured for a Tm170 radiation source of the invention having a 0.1 mm gold layer coating;

FIG. 6 (comparative) shows a photon spectrum of a Tm170 radiation source encased in a titanium capsule having wall thickness of about 0.05 mm; and

FIG. 7 shows the radiation dose from a radiation source comprising thulium 170 coated with 0.1 mm gold foil.

It should be noted that the embodiments exemplified in the Figures are not intended to be in scale and are in diagram form to facilitate ease of understanding and description.

### EXAMPLES

### Example 1

### Preparation of a Seed Comprising Thulium 170 Wrapped with a Gold Foil in a Titanium Canister

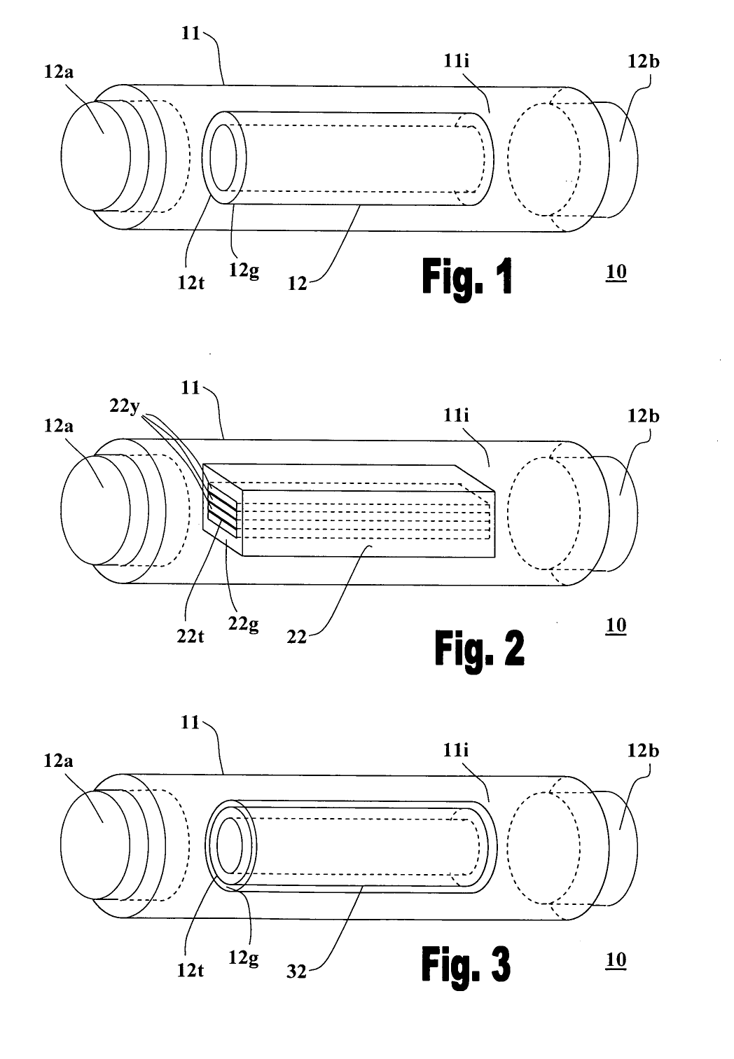

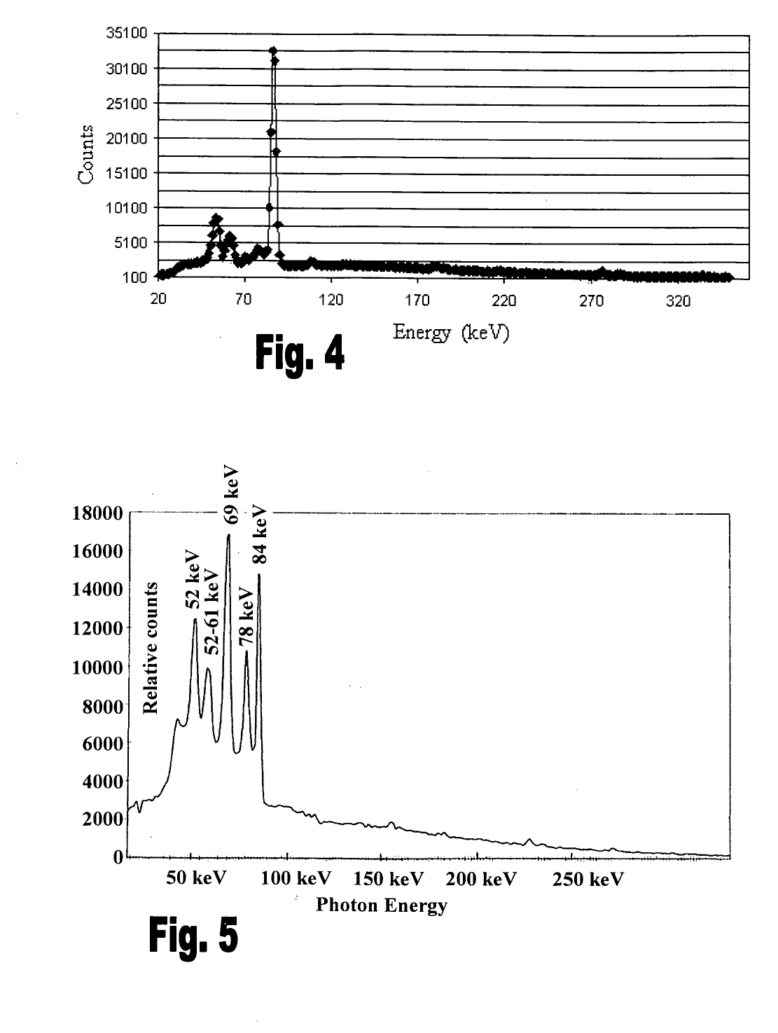

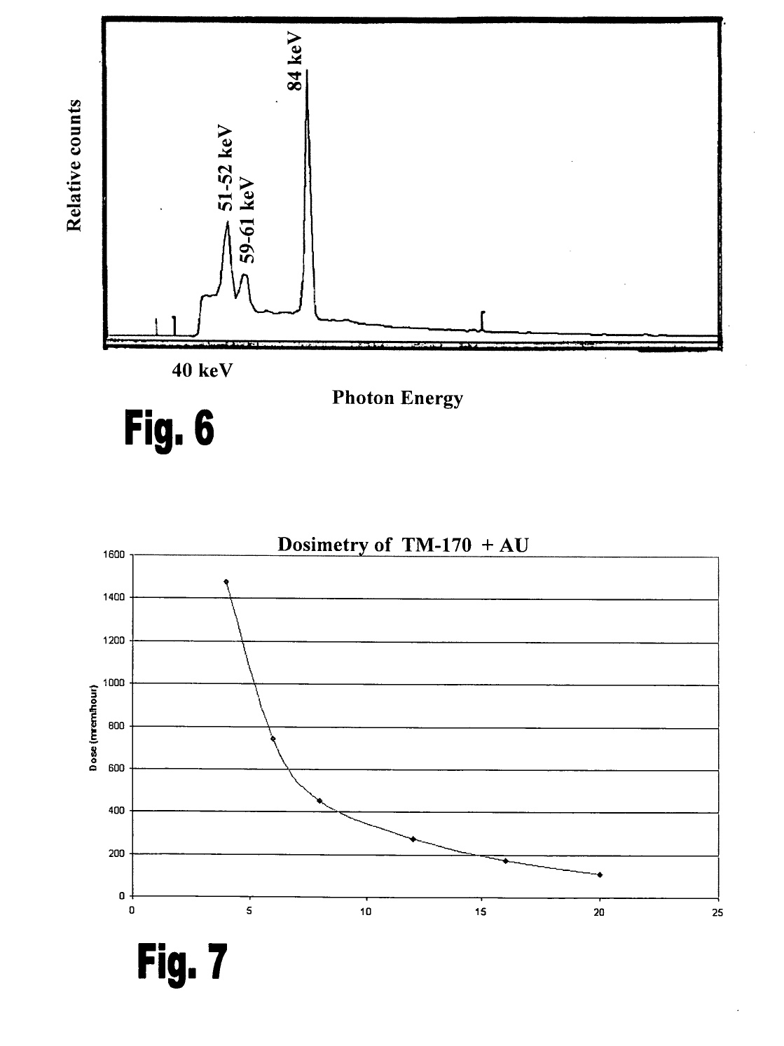

This example illustrates the preparation of the seed shown in FIG. 1.

Thulium 169 wire of 0.6 mm diameter was cut to obtain a section having a desired length (in the range between 1.0 mm and 5.0 mm). The thulium piece was then wrapped with a gold foil having thickness of about 0.1 mm (Goodfellow, England).

One end of a titanium tube (0.8 mm outer diameter, 0.7 inner diameter) was closed with a titanium plug. The plug is made of a titanium wire of diameter 0.7 mm (the inner diameter of the tube). A slice of 0.5 mm long of this wire was plugged into the titanium tube on one end and welded with a laser beam. The length of the titanium tube used depends on the length of the thulium piece+0.5 mm for a plug insertion on each end.

The gold coated thulium was inserted into the titanium tube through the open end. The second end of the tube was also plugged and sealed by welding. The titanium tube is now ready to be activated in order to convert the Tm-169 into Tm-170. The Activation time and flux level, which determine the source activity, were described hereinabove.

### Example 2

### Preparation of a Titanium Seed Comprising Thulium 170 Plated with Gold

This example illustrates the preparation of the seed shown in FIG. 1, wherein the thulium wire was plated with gold, applying a spattering technique. E5100 Polaron system was used for this purpose.

A gold foil, less than 1.0 mm thick on aluminum base was used as the gold source. The gold foil has a shape of a flat ring 10 cm diameter, 1 cm thick. This gold ring and the thulium seeds (thulium wire 0.6 mm diameter, 5 mm long) to be plated were placed in a vacuum chamber. The air was pumped out and argon gas was let into the chamber to a pressure of 0.1 to 0.2 Torr. The argon is kept flowing into the chamber during the gold plating to keep the pressure constant. A voltage of the order of 1-2 kV was applied between the gold electrode and the thulium wire pieces. The distance between the gold electrode and the thulium wire was about 50 mm. Ion current of about 20 mA flew between the gold electrode and the thulium wire, causing gold spattering from the gold electrode. Under such conditions, every five minutes the thickness of the gold layer increases in 75 Å. The growth of the gold coating was allowed to continue for 60 minutes. The gold-coated thulium is inserted into a titanium capsule and sealed as described in Example 1. The sealed capsule is delivered to a nuclear reactor to be activated as described hereinabove.

### Example 3

### Preparation of a Seed Comprising Thulium 170

This example illustrates the preparation of the seed shown in FIG. 1. However, the gold coating was applied externally onto the titanium canister subsequent to the activation of the seed in a nuclear reactor.

Thulium 169 wire of 0.6 mm diameter was cut to obtain a section having a desired length (in the range between 1.0 mm and 5.0 mm). One end of a titanium tube (0.8 mm outer diameter, 0.7 inner diameter) was closed with a titanium plug. The plug is made of a titanium wire of diameter 0.7 mm (the inner diameter of the tube). A slice of 0.5 mm long of this wire was plugged into the titanium tube on one end and welded with a laser beam. The length of the titanium tube used depends on the length of the thulium piece+0.5 mm for a plug insertion on each end. The thulium was inserted into the titanium tube through the open end. The second end of the tube was also plugged and sealed by welding. The titanium tube was transferred for activation in a nuclear reactor, in order to convert the Tm-169 into Tm-170. For considerations of safety, the seed is delivered within a quartz ampoule to the nuclear reactor. The seeds were activated at a neutron flux of 10¹³ n/s·cm² for 12 hours and were subsequently coated with a gold foil.

1) A low dose rate brachytherapy seed having activity in the range between 0.5 and 10 millicurie, comprising thulium-170 placed within a casing and at least one layer of a radiation emission modifying metal, said layer being provided either internally within the casing or on the outer surface thereof, wherein the radiation emission profile of the radiation source has the following features:
(i) the level of the beta radiation emitted is reduced in comparison with that generated by thulium 170;
(ii) the photon emission spectrum of said source exhibits one or more peaks in the region between 28 keV and 84 keV energy levels indicative of photons emitted by the layer(s) of the radiation emission modifying metal; and
(iii) the photon emission spectrum of said source comprises in addition to the discrete peaks also a continuous region indicative of the emission of braking radiation associated with the layer(s) of the radiation emission modifying metal.

2) A low dose rate brachytherapy seed according to claim 1, wherein the photon emission spectrum of the source exhibits one or more peaks in the region between 65 keV and 84 keV energy levels indicative of photons emitted by the layer(s) of the radiation emission modifying metal; and
(iv) the photon emission spectrum of the source displays a lower ratio between the intensities of the characteristic peaks at 84 keV and 52 keV energy levels, relative to the ratio observed in the corresponding spectrum of thulium 170.

3) A low dose rate brachytherapy seed according to claim 2, wherein the activity of the seed is less than 3 millicurie.

4) A low dose rate brachytherapy seed according to claim 1, wherein the photon emission spectrum of the source exhibits one or more peaks in the region between 28 keV and 40 keV energy levels indicative of photons emitted by the layer(s) of the radiation emission modifying metal.

5) A low dose rate brachytherapy seed according to claim 4, wherein the activity of the seed is less than 3 millicurie.

6) A low dose rate brachytherapy seed according to claim 1, wherein the radiation emission modifying metal is provided as a coating surrounding the thulium, said coating having a substantially uniform thickness.

7) A low dose rate brachytherapy seed according to claim 1 which emits isotropic beta radiation, the intensity of said radiation being in the range between 10% and 30% relative to the intensity of the beta radiation emitted by a corresponding non-coated thulium 170.

8) A low dose rate brachytherapy seed according to claim 1, wherein the thulium is provided as a solid cylinder, a rectangular parallelpiped or a tube-like member defined by a lateral surface of a cylinder.

9) A low dose rate brachytherapy seed according to claim 8, where the thulium is provided by a tube-like member defined by a lateral surface of a cylinder, the thickness of the walls of said tube-like member being in the range between 0.1 and 0.2 mm.

10) A low dose rate brachytherapy seed according to claim 3, wherein the radiation emission modifying metal is provided as a gold coating or platinum coating.

11) A low dose rate brachytherapy seed according to claim 5, wherein the radiation emission modifying element is provided by iodine, barium, lanthanum or cerium coating.

12) A low dose rate brachytherapy seed according to claim 1, wherein the casing is made of titanium.

13) A low dose rate brachytherapy seed according to claim 10, wherein the coating comprises gold, such that the ratio between the intensities of the characteristic peaks of thulium 170 at 84 keV and 52 keV energy levels is less than 2.5:1, and wherein said seed further exhibits at least one X-ray peaks at energy levels of about 65 to 84 keV.

14) A low dose rate brachytherapy method, which comprises implanting into or adjacent a tumor site of a patient a plurality of low dose rate thulium 170 brachytherapy seeds, wherein the thulium brachytherapy seed is as defined in claim 1.
